(19) United States
(12) Patent Application Publication
(10) Pub. No.: US 2011/0003778 A1
(43) Pub. Date: Jan. 6, 2011

(54) MINERALCORTICOID RECEPTOR ANTAGONISTS FOR THE TREATMENT OF ENDOMETRIOSIS

(75) Inventors: Carsten Moeller, Berlin (DE); Ulrike Kaufmann-Reiche, Berlin (DE); Anja Schmidt, Orange, NJ (US); Ulrike Fuhrmann, Berlin (DE); Oliver-Martin Fischer, Berlin (DE)

Correspondence Address: MILLEN, WHITE, ZELANO & BRANIGAN, P.C., 2200 CLARENDON BLVD., SUITE 1400, ARLINGTON, VA, 22201, US

(73) Assignee: Bayer Schering Pharma Aktiengesellschaft, Berlin (DE)

(21) Appl. No.: 12/529,447

(22) PCT Filed: Feb. 28, 2008
(86) PCT No.: PCT/EP08/52456

§ 371 Date: Feb. 22, 2010

(30) Foreign Application Priority Data
Mar. 2, 2007 (DE) 102007011105.5

(51) Int. Cl. A61K 31/57; A61K 31/585; A61K 31/56; A61P 35/04

(52) U.S. Cl. 514/170; 514/173; 514/171

(57) ABSTRACT

The present invention provides the use of mineralocorticoid receptor antagonists for preparing a medicament for the treatment of endometriosis.

The present invention relates in particular to an improved therapeutic composition for endometriosis which has a more favorable activity and side-effect profile than treatment therapies currently available.

Using a mineralocorticoid receptor antagonist, it is possible to threat endometriosis permanently without any loss of bone mass.

The present invention relates to the use of mineralocorticoid receptor antagonists for the treatment of endometriosis. In particular, the present invention relates to an improved therapeutic composition for the treatment of endometriosis having a better activity or side-effect profile than the treatment therapies currently available.

The clinical picture of endometriosis is a metastasis of endometrium tissue outside of the uterine cavity. These so-called endometriotic lesions innidiate at various locations of the peritoneal space, for example the intestinal wall, the ovary or rectovaginally, keeping the properties of their original tissue. Endometriosis is of essentially inflammatory character and affects 10-20% of women of reproductive age. The main symptoms of endometriosis are chronic abdominal pain, dysmenorrhoea, dyspareunia, dysuria, menstruation disorders and infertility. In most cases, the symptoms occur in combination. It is assumed that the lesions migrate via so-called retrograde menstruation via the uterine tube into the peritoneal space, where they innidiate.

Current therapeutic approaches for treating a diagnosed endometriosis are very limited. They include the use of GnRH agonists, androgens or else gestagens.

GnRH agonists suppress the endogenous production of estrogen by interfering with the hypothalamus/pituitary/ovary axis. As a result the estrogen concentrations are reduced to postmenopausal levels, with a concomitant reduction of the endometriosis-related symptoms. Androgens act in a similar manner, and in addition a direct action at the ovary is postulated. Owing to the side-effect profile, these two therapeutic approaches are only suitable for short-term applications (6-9 months).

GnRH agonists induce postmenopausal symptoms such as hot flushes and a reduction in bone mass. In the case of androgens, acne, weight increase and irreversible mood swings are additionally observed.

Currently, depot-MPA (medroxyprogesterone acetate) is the only gestagen approved for the treatment of endometriosis. Even after an application of only 6 months, the bone mass may be reduced. Therefore, under no circumstances should it be used for a period of more than 2 years (Physician Information for depo-subQ provera 104; Subcutaneous depot medroxyprogesterone acetate versus leuprolide acetate in the treatment of endometriosis-associated pain; P. G. Crosignani et al., Human Reproduction Vol. 21, No. 1 pp. 248-256, 2006).

EP 1 257 280 B1 indicates that micronized drospirenone may be suitable for the treatment of endometriosis: in [0045] of this publication it is described that compositions of drospirenone having a low content of estrogen or else without any estrogen may be suitable inter alia for the treatment of endometriosis. In the context of this disclosure, this is by virtue of the gestagenic properties of drospirenone. Thus, drospirenone has been described as being suitable for the treatment of endometriosis owing to its gestagenic activity. EP 1 257 280 B1 describes amounts of from 0.5 to 10 mg of drospirenone as being effective. EP 1 257 280 B1 does not say anything with respect to the duration of the treatment of endometriosis with drospirenone.

Endometriosis may furthermore be treated by surgical removal of the endometriotic lesions in a laparoscopic operation. However, the relapse rate after such an intervention is very high (25-30%). In such particularly difficult cases, hysterectomy, that is the complete removal of the uterus, is the final therapeutic option.

Thus, there are currently no long-term therapies for the indication of endometriosis available where side-effects, treatment efficiency and relapse rate are well balanced.

It is an object of the present invention to provide novel therapeutic approaches for the treatment of endometriosis having a better activity or side-effect profile than the treatment therapies currently available. In particular, the therapeutic approach of the invention should allow a permanent or long-term treatment of endometriosis.

This object is achieved by the present invention by a novel therapeutic approach for permanent treatment of endometriosis.

According to the invention, mineralocorticoid receptor antagonists are used for preparing a medicament for the treatment of endometriosis.

Here, the duration of the treatment with the mineralocorticoid receptor antagonist may extend over a period of at least 6 months, preferably even more than 24 months.

If the treatment is intended to be carried out over a period of less then 6 months, drospirenone is excluded as mineralocorticoid receptor antagonist.

To this end, the mineralocorticoid receptor antagonists can be used on their own or in combination with other substances.

In such combined applications, the use together with at least one compound from the group of the
gestagens,
so-called SERMs (Selective Estrogen Receptor Modulators),
SPRMs (Selective Progesterone Receptor Modulators),
combinations of gestagens and estrogens,
progesterone receptor antagonists,
estrogen receptor antagonists,
glucocorticoids,
estrogen receptor isotype-specific ligands (ER-β ligands),
androgens,
antiandrogens and
SARMs (Selective Androgen Receptor Modulators)
   is preferred.

The present invention also relates to the use of compounds which combine their anti-mineralocorticoid action with an action on other receptors for preparing a medicament for the treatment of endometriosis.

Here, preference is given to substances which are both anti-mineralocorticoid and show activity at the progesteron receptor, at the estrogen receptor, at the estrogen receptor beta, at the glucocorticoid receptor and/or at the androgen receptor, where this action at the last-mentioned receptors may be as an agonist, a partial agonist or else as an antagonist.

Mineralocorticoid receptor antagonists are substances which have binding affinity at the mineralocorticoid receptor and inhibit the action of the natural mineralocorticoid aldosterone. Examples which may be mentioned here are the compounds spironolactone, epleronone and drospirenone. The mineralocorticoid receptor antagonists to be used according to the invention should have a binding affinity at the mineralocorticoid receptor which corresponds approximately to that of aldosterone or is better than that of aldosterone. Here, only substances blocking aldosterone at its receptor, but not those inhibiting the endogenous production of aldosterone, are referred to as mineralcorticoid receptor antagonists.

Gestagens in the sense of the present invention are to be understood as meaning either natural progesterone itself or synthetic derivatives which, like progesterone itself, bind to the progesterone receptor and, at dosages higher then the ovulation-inhibiting dose, inhibit ovulation. Examples of synthetic derivatives which may be mentioned are drospirenone, gestodene, levonorgestrel, cyproterone acetate, desogestrel and 3-ketodesogestrel, norethisterone, norethisterone acetate and dienogest.

According to the invention, SERMs (Selective Estrogen Receptor Modulators) are compounds which, tissue-selectively, have either antiestrogenic or estrogenic action, and, for example, inhibit the action of estrogen at the uterus, but, at the bone, have neutral or estrogen-like action. Examples which may be mentioned are tamoxifen, raloxifen and basidoxifen.

In the context of the present invention, SPRMs (Selective Progesterone Receptor Modulators; sometimes also referred to as mesoprogestins) are to be understood as compounds which, in vivo at the progesterone receptor, have either agonistic or antagonistic activity. Like gestagens and progesterone receptor antagonists, SPRMs have high binding affinity to the progesterone receptor. However, compared to gestagens and progesterone receptor antagonists, SPRMs have different pharmacodynamic properties. The progesterone-agonistic activity, determined in vivo using customary biological tests (for example the McPhail test, Selye, H.), of the SPRM is the basic property of these compounds. However, this activity is less than that of progesterone. Pregnancy maintenance in ovarectomized pregnant rodents such as mice or rats cannot be achieved with SPRMs. On the other hand, SPRMs antagonize the action of progesterone. Here, however, the maximum antagonizing activity is less than that which can be induced by RU 486 or other pure progesterone antagonists. For further details, reference is made, for example, to WO 01/15679. Typical representatives of the class of compounds which may be mentioned are 4-[17β-methoxy-17α-(methoxymethyl)-3-oxoestra-4,9-dien-11β-yl]benzaldehyde (1E)-oxime, 4-[17β-methoxy-17α-(methoxymethyl)-3-oxoestra-4,9-dien-11β-yl]benzaldehyde (1E)-[O-(ethylamino)carbonyl]oxime and 4-[17β-methoxy-17α-(methoxymethyl)-3-oxoestra-4,9-dien-11β-yl]benzaldehyde (1E)-[O-(ethylthio)carbonyl]oxime.

SARMs (Selective Androgen Receptor Modulators) are substances which act tissue-selectively as androgens or anti-androgens, for example as anti-androgens in the skin, but have no or a positive effect on libido.

Combinations of gestagens and estrogens are active compound combinations which are found in oral contraceptives known per se, for example Yasmin, Femovan, Triquilar, Marvelon, YAZ etc.

Progesterone receptor antagonists are compounds which inhibit the action of progesterone at its receptor. Examples which may be mentioned are RU 486, Onapriston and 11β-(4-acetylphenyl)-17β-hydroxy-17α-(1,1,2,2,2-pentafluoroethyl)estra-4,9-dien-3-one (WO 98/34947).

Estrogen receptor antagonists are compounds which block the action of estrogen at its receptors (the estrogen receptor α and the estrogen receptor β). An estrogen receptor α-blocking compound which may be mentioned is, for example, 7α-[9-(4,4,5,5,5-pentafluoropentylsulphinyl]-n-nonyl]estra-1,3,5(10)-trien-3,17β-diol.

Glucocorticoids are compounds which induce agonistic action at the glucocorticoid receptor, thus acting as immunosuppressants. An example which may be mentioned is dexamethasone.

According to the invention, the mineralcorticoid receptor antagonists are used in the following dosages:

spironolactone 10 to 500 mg/d and person, administered once or as 2 half-doses, preferably 25 to 250;
eplerenone 10 to 500 mg/d and Person, administered once or as 2 half-doses, preferably 25 to 250,
drospirenone 1 to 5 mg, preferably 2 to 4 mg.

Other mineralcorticoid receptor antagonists are used in dosages of equivalent activity, i.e. dosages which give a result in the treatment of endometriosis which is comparable to that of the amounts of the compounds stated above.

If the mineralcorticoid receptor antagonists are used together with another compound (see above), these compounds are used in the dosage ranges already described for these other compounds.

The mineralcorticoid receptor antagonists and, if appropriate, further compounds to be used are formulated in a manner known per se to the person skilled in the art.

The classic physiological function of aldosterone as effector of the renin/angiotensin/aldosterone system is the regulation of the extracellular volume and the homeostasis of the potassium metabolism. However, the activation of the mineralocorticoid receptor may, in various tissues where the mineralocorticoid receptor is expressed, such as the brain, the heart or the blood vessels, result in pathological symptoms. Examples which may be mentioned here are hypertension, vascular disorders, renal dysfunction, migraine, neuropathy, retinopathy, baroreceptor dysfunctions, liver disorders and edema. It is postulated that these harmful effects of aldosterone are caused by the mineralocorticoid receptor in non-classic target organs, for example the blood vessels (Rudolph et al., 2004).

The action of the diuretics spironolactone and epleronone is due to the action of aldosterone at the target organ kidney: in the presence of aldosterone they increase renal sodium excretion and lower the excretion of potassium. The result is an increased elimination of water. They are therefore used for treating hypertension (Weinberger et al., 2002; Weinberger et al., 2005). However, the inhibition of aldosterone with the aid of substances such as epleronone also has a protective effect on various target organs without any accompanying measurable lowering of blood pressure, or without any diuretic action (Rudolph et al., 2004). In an animal model it was possible to demonstrate, for example, that epleronone inhibits the aldosterone-mediated initial inflammatory damage of blood vessels and myocardial fibrosis in models of myocardial infarction (Rocha et al., 2002a; Rocha et al., 2002b). More recent in vitro studies have shown that spironolactone may have an effect on the biosynthesis of certain cytokines in cells of the immune system (Mikkelsen et al., 2006).

A physiological or pathophysiological function of aldosterone or the mineralocorticoid receptor in the onset and progression of endometriosis has hitherto not been described.

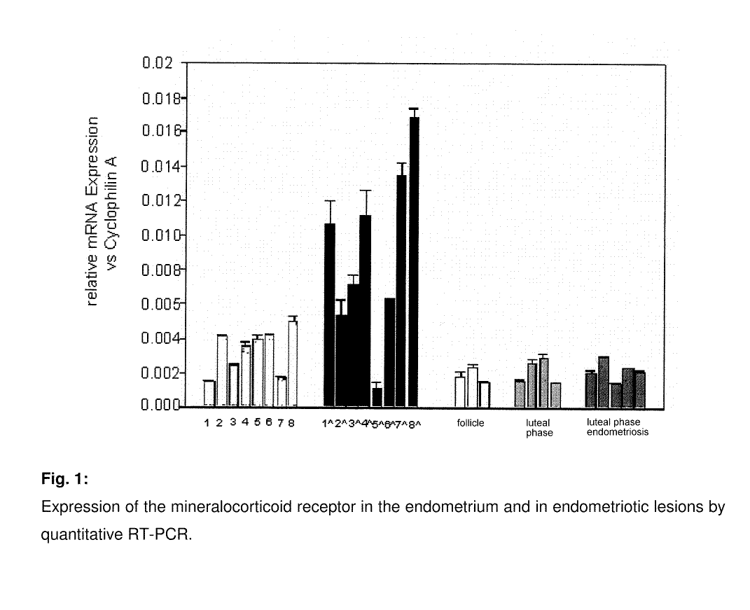

According to the invention, it has now been found that, surprisingly, in endometriosis the mineralocorticoid receptor has a modified expression profile. This can be determined, for example, by quantitative RT-PCR where the mRNA content is measured compared to a reference gene. In the pathological tissue, the endometriotic lesions, the mineralocorticoid receptor shows a 2- to 3-times higher mRNA content compared to healthy tissue, the endometrium of the uterus (see FIG. 1). When the expression of the mineralocorticoid receptor in the endometrium is compared for different stages of the menstrual cycle, changes in the levels of expression (mRNA content) are not evident (FIG. 1). This suggests that in the normal physiology of the endometrium, i.e. in the healthy endometrium, aldosterone and the mineralocorticoid receptor have no regulating function.

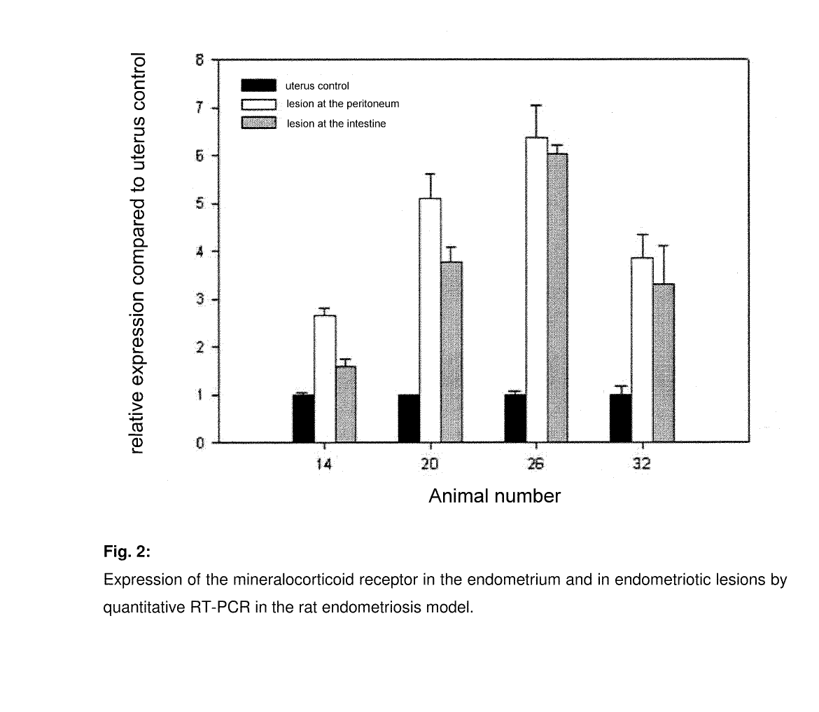

It was furthermore possible according to the invention to show that the expression of the mineralocorticoid receptor in endometriotic lesions in a rodent (in this case rats) endometriosis model is likewise increased, here, too, compared to the endometrium of the uterus (FIG. 2). Thus, the role of the mineralocorticoid receptor in endometriosis appears to be consistent for different species.

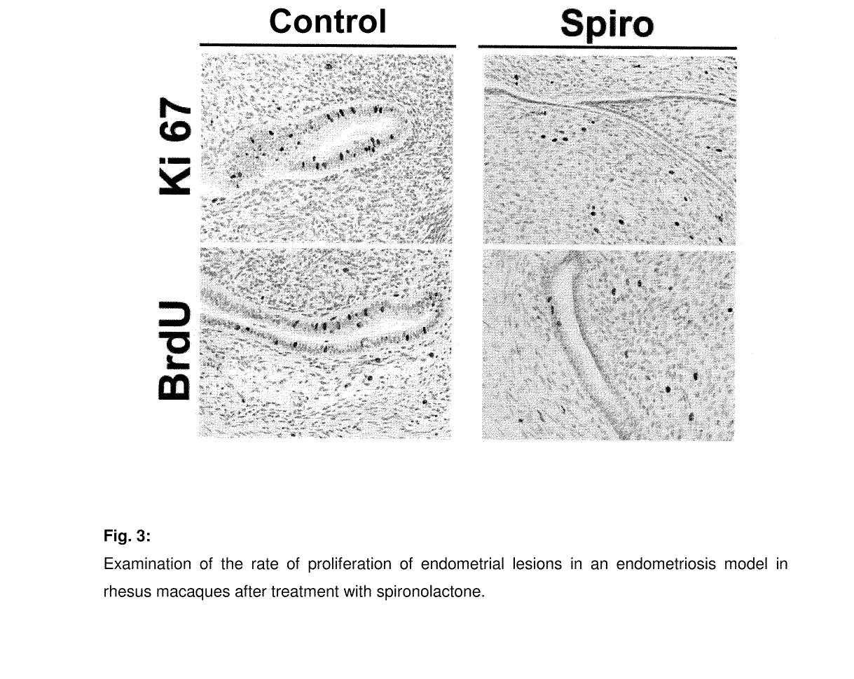

Moreover, according to the invention it has been found that substances which inhibit the action of aldosterone at the mineralocorticoid receptor have, in a primate model, an effect on endometriosis which suggests a positive effect in the treatment of endometriosis in humans (FIG. 3). In this test, the mineralocorticoid receptor antagonist spironolactone, a standard compound from this class of active compounds, was used.

Based on these observations, which are described in more detail in Examples 1 to 3, it has to be assumed that treatment of female patients suffering from endometriosis with a mineralocorticoid receptor antagonist will result in a reduction of symptoms since treatment in the primate model resulted both in a reduction of the size of the endometriotic lesions and in an inhibition of the proliferation of these lesions.

One embodiment of the invention provides the use of a combination of mineralocorticoid receptor antagonists and at least one compound from the group of the gestagens, progesterone receptor antagonists, glucocorticoids, a combination of gestagens and estrogens, SERMs, SPRMs and estrogen receptor isotype-specific ligands (selective ER β agonists).

The use of compounds from these classes of active compounds for the treatment of endometriosis is already known. There are suggestions in the literature of treating endometriosis with SPRMs and progesterone receptor antagonists (Kettel et al., 1996), and the use of gestagens and combinations of gestagens and estrogens for the same purpose has also been described (Rodgers and Falcone, 2008). Furthermore, preclinical animal models suggest that estrogen receptor isotype-specific ligands may have a positive effect in endometriosis (Harris et al., 2005). In addition, it is known that glucocorticoids may act as immunosuppressants and may thus have a positive effect on the progression of endometriosis. However, a safe long-term use in women has not been described or disclosed for any of the approaches described above. A combination of a mineralocorticoid receptor antagonist with one of these active principles of the prior art should, firstly, have an additive effect in the action and, secondly, also allow long-term use. In the combination therapy, the substances administered in addition to the mineralocorticoid receptor antagonists should be used in the pharmacologically effective dosages already described, SPRMs and progesterone receptor antagonists, for example, in a dosage range of from 1 mg to 100 mg per woman and day, and glucocorticoids in a dosage range of from 0.01-2 mg/kg and day.

Particularly suitable in this case is the use of mineralocorticoid receptor antagonists which simultaneously exhibit either gestagenic activity, or have progesterone receptor-antagonistic activity or glucocorticoid activity or estrogen receptor isotype-specific activity.

In all cases, the mineralcorticoid receptor antagonist can be administered continuously or discontinuously.

Preference is given to treatment protocols where the mineralcorticoid receptor antagonist is administered daily.

Particular preference is given to treatment protocols where the mineralcorticoid receptor antagonist is administered daily in oral form.

Combinations of mineralcorticoid receptor antagonists with gestagens, progesterone receptor antagonists, combinations of estrogens with gestagens, estrogen receptor isotype-specific ligands, with SERMs, SPRMs and glucocorticoids can likewise be administered continuously or discontinuously.

Particular preference is given here to combinations of mineralcorticoid receptor antagonists and gestagens, mineralcorticoid receptor antagonists and progesterone receptor antagonists and mineralcorticoid receptor antagonists and combinations of estrogens and gestagens.

For combinations of mineralcorticoid receptor antagonists with gestagens, preference is given to treatment protocols where the gestagens are administered continuously at a daily dosage.

In the case of a combination of mineralcorticoid receptor antagonists with combinations of estrogens and gestagens, these can be administered either continuously or discontinuously. In the case of discontinuous administration, preference is given in particular to 21/7 or 24/4 cycles (21 days of administration of the combination followed by 7 days of rest, either by administration of a placebo or by a treatment break; correspondingly for 24/4).

Preferred gestagens are the compounds medroxyprogesterone acetate, cyproterone acetate, levonorgestrel, norgestimate, desogestrel, gestodene, dienogest. In such combinations, the preferred estrogen is the compound ethynylestradiol.

In the case of mineralcorticoid receptor antagonists which additionally exhibit activity at other receptors, particular preference is given to compounds which are additionally active at the progesterone receptor, or which are additionally active as antagonists at the androgen receptor. Particular preference is given here to mineralcorticoid receptor antagonists which simultaneously exhibit gestagenic activity.

These can be administered continuously or discontinuously.

Particular preference is given here to treatment protocols comprising a daily oral administration. Mineralcorticoid receptor antagonists which simultaneously have gestagenic activity can also be administered in combination with estrogens.

A particularly preferred estrogen is ethynylestradiol.

Such combinations may be administered either continuously (daily) or in a discontinuous treatment protocol.

Particularly preferred as such discontinuous treatment protocols are those which provide an administration for 24 days followed by a break of 4 days or those which provide a treatment of 21 days followed by a break of 7 days. Breaks in these treatment protocols can be either by administration of placebos, or by simply interrupting the treatment.

The invention is illustrated in more details by Examples 1 to 3 below and the accompanying FIGS. 1 to 3; however, the invention is not limited to these examples.

### EXAMPLE 1

The expression of the mineralocorticoid receptor in human endometriotic lesions was compared to that in normal human endometrium. To this end, from appropriate tissue biopsies, the mineralcorticoid receptor NA was isolated by phenol/chloroform extraction, transcribed via reverse transcription into cDNA and examined by TaqMan-RT-PCR analysis according to the standard protocol for the relative content of mineralcorticoid receptor/mineralcorticoid receptor NA. The TaqMan probes used were commercially available primer/probe combinations from BD Biosciences for the mineralcorticoid receptor and the control gene cyclophilin A.

### FIG. 1:

Expression of the mineralocorticoid receptor in the endometrium and in endometriotic lesions by quantitative RT-PCR.

Compared to healthy endometrium, the endometriotic lesions show a considerably elevated expression (compare samples 1-8 to samples 1*-8*, 1-8 correspond to eight different female patients, the endometriotic lesions are marked with asterisks). The phase of the menstrual cycle has no effect on the level of expression in the endometrium (compare follicle phase to luteal phase). Moreover, there is no difference in the expression of the mineralcorticoid receptor in the eutopic endometrium between healthy subjects and endometriosis patients (compare luteal phase to luteal phase endometriosis). All this suggests a role of the mineralcorticoid receptor in the pathology of endometriosis.

Compared to normal endometrium (1-8, left, first group from the left, white) the MR in the endometriotic lesions exhibits an mRNA expression with is elevated by a factor of 2-3 (1*-8*, second group from the left, black). When healthy endometrium is compared over different stages of the menstrual cycle, a difference in expression levels is not evident (follicle phase compared to luteal phase; third group from the right, white, compared to the second group from the right, light-gray). The MR expression in the endometrium of endometriosis patients does not differ from that of healthy female patients (luteal phase endometriosis compared to luteal phase, first group from the right, gray, compared to 2. group from the right, light-gray).

### Beispiel 2:

The expression of the mineralocorticoid receptor in endometriotic lesions was compared to that in normal endometrium using a rodent model for endometriosis (Matsuzaki et al., 2004). To this end, endometriosis tissue was removed from the estrus of adult female rats and transplanted in an autologous transplantation into two positions in the peritoneal space, firstly the wall of the colon, and secondly the peritoneum. In this experimental approach, the reattached or closed uterus (after removal of the endometrium) serves as control tissue. Four week after placing of the lesions, the lesions and the control tissue were removed and the mineralcorticoid receptor NA was isolated from these tissue biopsies by phenol/chloroform extraction, transcribed via reverse transcription into cDNA and finally in a TaqMan-RT-PCR analysis according to the standard protocol examined for the relative content of mineralcorticoid receptor/mineralcorticoid receptor NA. The lesions show a considerably increased expression of the mineralocorticoid receptor compared to the normal uterus (see FIG. 2). Accordingly, the increased expression of the mineralcorticoid receptor is conserved even in this model, which underlines the pathological function of the mineralcorticoid receptor in this indication.

### FIG. 2:

Expression of the mineralocorticoid receptor in the endometrium and in endometriotic lesions by quantitative RT-PCR in the rat endometriosis model.

The lesions show a considerably increased expression of the mineralocorticoid receptor compared to the control tissue (compare in each case the expression of uterus control, lesion at the peritoneum and lesion at the intestine; in total, 4 animals were used for this experiment (numbers 14, 20, 26 and 32).

In all four animals examined (animals numbers 14, 20, 26, 32) both lesions (light-gray, lesion at the peritoneum, and gray, lesion at the intestine) show increased expression of the MR compared to the uterus control (black).

### EXAMPLE 3

The activity of the mineralcorticoid receptor antagonist spironolactone was tested in an endometriosis model in non-human primates. Non-human primates are the only species apart from man where endometriosis occurs spontaneously under natural conditions. They are therefore the most relevant model for examining this disorder. In this model, endometrium tissue is, for reasons of reproducibility after ovarectomy, transplanted subcutaneously and interperitoneally into rhesus macaques in an autologous transplantation and, after an artificially induced menstrual cycle, treated for 60 days either with physiological concentrations of estradiol (control) or with a combination of estradiol and spironolactone (30 mg/kd/day). In this procedure, the animals are given a depot for estradiol substitution which keeps the serum level at 80 to 100 pg/ml. In this manner, it is ensured that the endometriotic lesions are exposed to physiological concentrations of estradiol. By virtue of the depot, interindividual variations of the hormone concentrations and their effect on the results can be excluded. After the treatment, the size of the endometrium lesions was determined, and the proliferation of these lesions was detected by a histological analysis. Compared to control animals, the spironolactone-treated animals show a significant reduction of the endometriotic lesions (Table 1). Both staining of the mitotic antigen Ki67 and the corresponding treatment with bromodeoxyuridine (BrDU) show a reduction of proliferation in the abdominal lesions (see FIG. 3). Accordingly, the mineralcorticoid receptor antagonist spironolactone has a positive effect on endometriosis genesis.

TABLE 1
Uterus weight and weights of endometrial lesions
after 60 days of treatment in a macaque model.

| | E₂ | E₂ + Spirolactone |
|---|---|---|
| | (n = 4) | (30 mg/kg/d) (n = 4) |
| Uterus wt (g) | 3.75 + 0.496 | 5.01 + 0.99 |
| Subcutaneous | 0.039 + 0.008 | 0.02 + 0.004 |
| lesion wt (g) | | |
| Intraabdominal | 0.068 + 0.11 | 0.049 + 0.009 |
| lesion wt (g) | | |

In an autologous transplantation, a plurality of lesions from endometrium tissue were implanted subcutaneously and intraperitoneally/abdominally into rhesus macaques. After an artificially induced menstrual cycle, the animals were treated for 60 days either with estradiol (control) or with estradiol and spironolactone. After treatment with spironolactone, both types of lesions showed a reduction in weight. The treatment with spironolactone has no significant effect on the uterus as such.

### FIG. 3:

Examination of the rate of proliferation of endometrial lesions in an endometriosis model in rhesus macaques after treatment with spironolactone.

Both immunohistological staining with Ki67 (a marker for mitosis) and the corresponding treatment with bromodeoxyuridine (BrDU) show that proliferation in the secretory cells of the lesions is strongly reduced as a result of the treatment of the endometrial transplants.

Both detection of cell division in the endometriotic lesions by Ki67 (top row) and by incorporation of BrDU (bottom row) show a reduced value in the animals treated with spironolactone (control, left, compared to spironolactone, right).

### REFERENCES

Crosignani, P. G., Luciano, A., Ray, A. and Bergqvist, A. (2006). Subcutaneous depot medroxyprogesterone acetate versus leuprolide acetate in the treatment of endometriosis-associated pain. Human Reproduction 21, 248-256.
Harris H A, Bruner-Tran K L, Zhang X, Osteen K G, Lyttle C R (2005). A selective estrogen receptor-β agonist causes lesion regression in an experimentally induced model of endometriosis. Human Reproduction 20:936-941
Kettel, L. M., Murphy, A. A., Morales, A. J., Ulmann, A., Baulieu, E. E., Yen, S. S. (1996). Treatment of endometriosis with the antiprogesterone mifepristone (RU486). Fertil Steril 65(1), 23-8.
Matsuzaki, S., Canis, M., Darcha, C., Dallel, R., Okamura, K. and Mage, G. (2004). Cyclooxygenase-2 selective inhibitor prevents implantation of eutopic endometrium to ectopic sites in rats. Fertil Steril 82, 1609-15.
Mikkelsen, M., Sonder, S. U., Nersting, J. and Bendtzen, K. (2006). Spironolactone induces apoptosis in human mononuclear cells. Association between apoptosis and cytokine suppression. Apoptosis 11, 573-9.
Rocha, R., Martin-Berger, C. L., Yang, P., Scherrer, R., Delyani, J. and McMahon, E. (2002a). Selective aldosterone blockade prevents angiotensin II/salt-induced vascular inflammation in the rat heart. Endocrinology 143, 4828-36.
Rocha, R., Rudolph, A. E., Frierdich, G. E., Nachowiak, D. A., Kekec, B. K., Blomme, E. A., McMahon, E. G. and Delyani, J. A. (2002b). Aldosterone induces a vascular inflammatory phenotype in the rat heart. Am J Physiol Heart Circ Physiol 283, H1802-10.
Rodgers, A. K. and Falcone, T. (2008) Treatment strategies for endometriosis. Expert Opin Pharmacother 9 (2), 243-55.
Seyle, H., Textbook of Endocrinology 1947, 345-346.
Rudolph, A. E., Rocha, R. and McMahon, E. G. (2004). Aldosterone target organ protection by eplerenone. Mol Cell Endocrinol 217, 229-38.
Weinberger, M. H., Roniker, B., Krause, S. L. and Weiss, R. J. (2002). Eplerenone, a selective aldosterone blocker, in mild-to-moderate hypertension. Am J Hypertens 15, 709-16.
Weinberger, M. H., White, W. B., Ruilope, L. M., MacDonald, T. M., Davidson, R. C., Roniker, B., Patrick, J. L. and Krause, S. L. (2005). Effects of eplerenone versus losartan in patients with low-renin hypertension. Am Heart J 150, 426-33.
Physician Information for depo-subQ provera 104

1. A method for the treatment of endometriosis comprising administering to a patient an effective amount of a mineralcorticoid receptor antagonist over a period of at least 6 months.

2. The method as claimed in claim 1, wherein said mineralcorticoid receptor antagonist is administered over a period of at least 24 months.

3. A method for for the treatment of endometriosis, comprising administering to a patient an effective amount of a mineralcorticoid receptor antagonist other than drospirenone.

4. The method according to claim 1, wherein said mineralcorticoid receptor is spironolactone.

5. The method according to claim 1, wherein said mineralcorticoid receptor is eplerenone.

6. The method according to claim 1, wherein said mineralcorticoid receptor is drospirenone.

7. The method according to claim 1, wherein the mineralcorticoid receptor antagonists combines its anti-mineralocorticoid action with the action at other receptors.

8. The method according to claim 1, further comprising administering to said patient at least one compound from the group of the gestagens, the SERMs (Selective Estrogen Receptor Modulators), SPRMs (Selective Progesterone Receptor Modulators), combinations of gestagens and estrogens, progesterone receptor antagonists, estrogen receptor antagonists, glucocorticoids, estrogen receptor isotype-specific ligands (ER-β ligands), androgens, antiandrogens and SARMs (Selective Androgen Receptor Modulators).

9. The method according to claim 8, wherein said at least one compound is a glucocorticoid.

10. The method according to claim 8, wherein said at least one compound is a progesterone receptor antagonist.

11. The method according to claim 8, wherein said at least one compound is an estrogen receptor antagonist.

12. The method according to claim 8, wherein said at least one compound is an estrogen receptor isotype-specific ligand (ERβ ligands).

13. The method according to claim 8, wherein said at least one compound is a gestagen and an estrogen.

14. The method according to claim 13, wherein said estrogen is ethynylestradiol.

15. The method according to claim 1, wherein the administration of the mineralcorticoid receptor antagonist is carried out continuously (daily).

16. The method according to claim 1, wherein the administration of the mineralcorticoid receptor antagonist is carried out discontinuously.

17. The method according to claim 1, wherein the mineralcorticoid receptor antagonist is administered in oral form.
